(19) United States
(12) Patent Application Publication
(10) Pub. No.: US 2007/0093789 A1
(43) Pub. Date: Apr. 26, 2007

(54) Iontophoresis apparatus and method for delivery of angiogenic factors to enhance healing of injured tissue

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Tokyo (JP)

(21) Appl. No.: 11/540,934

(22) Filed: Sep. 29, 2006

Related U.S. Application Data
(60) Provisional application No. 60/722,849, filed on Sep. 30, 2005.

(51) Int. Cl. A61K 9/22; A61M 31/00

(52) U.S. Cl. 604/890.1; 604/501

(57) ABSTRACT

An iontophoresis device includes an active electrode assembly including an active electrode element and at least one active agent reservoir. Active agents include one or more angiogenic growth factors suitable to promote or enhance healing or repair of injured or damaged tissue.

### CROSS-REFERENCE TO RELATED APPLICATION

This application claims the benefit under 35 U.S.C. § 119(e) of U.S. Provisional Patent Application No. 60/722,849 filed Sep. 30, 2005, where this provisional application is incorporated herein by reference in its entirety.

### BACKGROUND OF THE INVENTION

1. Field of the Invention

The present disclosure generally relates to the field of iontophoresis, and, more particularly, to the effective delivery of angiogenic factors to a biological interface to enhance healing of injured or damaged tissue.

2. Description of the Related Art

Iontophoresis employs an electromotive force and/or current to transfer an active agent (e.g., a charged substance, an ionized compound, an ionic drug, a therapeutic, a bioactive agent, and the like), to a biological interface (e.g., skin, mucous membrane, and the like), by using a small electrical potential to an electrode proximate an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power source. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent. Iontophoresis may be advantageously used to enhance or control the delivery rate of the active agent. The active agent may be stored in a reservoir such as a cavity. See, e.g., U.S. Pat. No. 5,395,310. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of the oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their ability to deliver drugs across various biological interfaces including, for example, tissue barriers. For example, it may be desirable to have novel approaches for overcoming the poor permeability of skin.

Angiogenesis, the process of forming new blood vessels, plays a significant role in physiological events such as wound healing and tissue remodeling. After injury or insult to tissue, angiogenesis is instrumental in restoring blood flow to the site of injury or insult in order to supply a variety of nutrients and factors necessary for healing and rebuilding to occur. Angiogenesis involves the proliferation, migration and assembly of endothelial cells into functional blood vessels from pre-existing blood vessels. Angiogenesis is stimulated by any of a variety of growth factors and is inhibited by any of a variety of inhibitors. Such factors are primarily proteins or polypeptides. In the healthy body under usual conditions, the angiogenic process is continually controlled by maintenance of an optimal balance between growth factors and inhibitors of angiogenesis.

After injury or insult at a particular site in a body, the injured or damaged tissue produces and releases angiogenic growth factors, which diffuse into the local tissue, thus leading to a localized excess of angiogenic growth factors over inhibitors of angiogenesis, allowing for the healing, repair and rebuilding of the injured or damaged tissue to occur. The growth factors bind to specific receptors on endothelial cells of nearby pre-existing blood vessels, activating the endothelial cells to produce enzymes to initiate the series of angiogenic events, ultimately leading to the formation of new blood vessels and the growth of new, healthy tissue.

Angiogenic growth factors, in addition to being supplied to the site of the injury or insult by normal physiological processes, may be administered therapeutically to assist in the healing and repair processes. Use of agents, such as angiogenic growth factors, to stimulate growth of new blood vessels to promote and/or enhance healing or repair of injured or damaged tissue have been proposed for treatment of a variety of conditions, for example, cardiac ischemia, chronic wounds, peripheral vascular disease, and stroke. PDGF-BB, for example, has been commercialized to promote healing in chronic wounds. Therapeutic administration of one or more angiogenic growth factors may be particularly useful in treating injury or insult in populations in which healing or repair is impaired, for example, in the elderly and/or in those with diabetes. Such populations, in particular, may suffer from bedsores or other ulcerative conditions. Topical application or systemic administration of angiogenic growth factors may be used. Both methods of administration have certain disadvantages. Topically applied factors or compositions, for example, are locally applied such that active agent may reach only certain superficial sites with which the topical application is in contact. An active agent may reach sites other than those with which direct contact has been made, whether superficial or internal, only passively, for example by diffusion, or not at all. Alternatively, when factors are administered systemically, they are delivered throughout the body. Administration is not localized. In part due to dilution of the active agent throughout the circulatory system, excessive amounts of agent will likely be required. Furthermore, certain angiogenic factors have significant undesirable side effects which may limit their utility when administered systemically, including accelerated growth of cells other than those targeted, for example, tumor cells.

The present disclosure is directed to overcoming one or more of the shortcomings set forth above, and to providing further related advantages.

### BRIEF SUMMARY OF THE INVENTION

In one aspect, the present disclosure is directed to an iontophoretic delivery device for providing transdermal delivery of one or more therapeutic active agents to a biological interface. The iontophoretic delivery device includes an active electrode assembly including at least one active electrode element and at least one active agent reservoir.

In certain embodiments, the at least one active electrode element is operable to provide an electromotive force for driving the therapeutic active agent from the at least one active agent reservoir to the biological interface.

In some aspects, the present disclosure is directed to an iontophoretic device, and to a method of use thereof, for providing transdermal delivery to a biological interface of one or more active agents useful to promote and/or enhance healing or repair of injured or damaged tissue.

In certain aspects, the present disclosure is directed to a method using an iontophoretic delivery device for delivering one or more angiogenic growth factors, or a composition thereof, or an injured or damaged tissue of a subject to promote or enhance healing or repair of the injured or damaged tissue. In at least one aspect, a method for promoting or enhancing healing or repair of an injured or damaged tissue comprises positioning an iontophoretic device on an area of injured or damaged tissue, switching the device on to supply an electromotive force or current, and delivering one or more angiogenic growth factors, or a composition thereof, to or into the injured or damaged tissue.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements and have been solely selected for ease of recognition in the drawings.

FIG. 1A is a top, front view of a transdermal drug delivery system according to one illustrated embodiment.

FIG. 1B is a top, plan view of a transdermal drug delivery system according to one illustrated embodiment.

FIG. 2A is a schematic diagram of the iontophoresis device of FIGS. 1A and 1B comprising active and counter electrode assemblies according to one illustrated embodiment.

FIG. 2B is a schematic diagram of the iontophoresis device of FIG. 2A positioned on a biological interface, with an optional outer release line removed to expose the active agent, according to another illustrated embodiment.

### DETAILED DESCRIPTION OF THE INVENTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with iontophoresis devices, including but not limited to voltage and/or current regulators, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” or “another embodiment” means that a particular referent feature, structure, or characteristic described in connection with the embodiment is included in at least one embodiment. Thus, the appearances of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoresis device including “an electron element” includes a single electrode element, or two or more electrode elements. It should also be noted that the term “or” is generally employed in its sense including “and/or” unless the content clearly dictates otherwise.

As used herein and in the claims, the term “membrane” means a boundary, a layer, a barrier or material, which may or may not be permeable. The term “membrane” may further refer to an interface. Unless specified otherwise, membranes may take the form of a solid, liquid, or gel, and may or may not have a distinct lattice, non-cross-linked structure, or cross-linked structure.

As used herein and in the claims, the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion, opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different than the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non-cross-linked gel, a jelly-like state, and the like. In some embodiments, the gel matrix may result from a three dimensional network of entangled macromolecules (e.g., cylindrical micelles). In some embodiments, a gel matrix may include hydrogels, organogels, and the like. Hydrogels refer to three-dimensional networks of, for example, cross-linked hydrophilic polymers in the form of a gel and substantially composed of water. Hydrogels may have a net positive or negative charge, or may be neutral.

As used herein and in the claims, the term “reservoir” means any form or mechanism to retain an element, compound, pharmaceutical composition, diagnostic composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and or current into the biological interface. A reservoir may also retain an electrolyte solution.

As used herein and in the claims, “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like), non-pharmaceuticals (e.g., a cosmetic substance, and the like), diagnostic agents, a vaccine, an immunological agent, a local or general anesthetic or painkiller, an antigen or a protein or a peptide, such as insulin, a chemotherapy agent, or an anti-tumor agent.

In some embodiments, the term “active agent” refers to the active agent itself, as well as its pharmacologically active salts, pharmaceutically or diagnostically acceptable salts, pro-drugs, metabolites, analogs, and the like. In some further embodiments, the active agent includes at least one ionic, cationic, ionizable, and/or neutral therapeutic drug and/or pharmaceutically acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. For instance, an active agent having an amino group can typically take the form of an ammonium salt in solid state and dissociate into a free ammonium ion (NH₄⁺) in an aqueous medium of appropriate pH. Other active agents may have functional groups that are readily convertible to a negative ion or can dissociate into a negatively charged ion and a counter ion in an aqueous medium. Yet other active agents may be polarized or polarizable, that is, exhibiting a polarity at one portion relative to another portion.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from growth factors, for example angiogenic growth factors, analgesics, anesthetics, vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of angiogenic growth factor active agents include platelet-derived growth factor-BB (PDGF-BB), platelet-derived endothelial growth factor (PD-EDGF), angiogenin, angiopoietin-1, angiotropin, acidic fibroblast growth factor (aFGF), basic fibroblast growth factor (bFGF), Del-1, follistatin, granulocyte colony-stimulating factor (G-CSF), hepatocyte growth factor (HGF)/scatter factor (SF), interleukin-8 (IL-8), leptin, midkine, placental growth factor, pleiotrophin, progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF). Many such growth factors are commercially available and/or may be prepared by methods known in the art.

Further non-limiting examples of active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate, and other 5-hydroxytryptamine 1 receptor subtype agonists; resiquimod, imiquimod, and similar TLR 7 and TLR 8 agonist and antagonists; domperidone, granisetron hydrochloride, ondansetron, and other such anti-emetic drugs; zolpidem tartrate and similar sleep inducing agents; L-DOPA and other anti-Parkinson's medications; aripiprazole, olanzapine, quetiapine, risperidone, clozapine, and ziprasidone, as well as other neuroleptica; diabetes drugs, such as exenatide; as well as peptides and proteins for treatment of obesity and other maladies.

Additional non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, “antigen” or “antigenic” or “antigenicity” refers to a protein, polypeptide or carbohydrate, and the like, that is recognized by the body as foreign and that stimulates the immune system to produce an antibody; as used herein and in the claims, “antigenic determinant”, also commonly referred to as “epitope,” refers to a specific area or structure (that is, an “antigenic site”) on the surface of an antigen that can cause an immune response, thus stimulating production of an antibody that can recognize and bind to the antigenic site or to structurally related antigenic sites. As used herein and in the claims, an “antigenic portion” of an antigen is a portion that is capable of reacting with serum obtained from an individual infected with an organism from which the antigen is derived or with the antigen itself.

As used herein and in the claims, a polypeptide comprising an antigenic determinant that is “similar to” an antigenic determinant located on a specified antigen refers to a polypeptide that elicits an immune response comparable to that elicited by the specified antigen.

As used herein and in the claims, the term “immunogen” or “immunogenicity” refers to any agent that elicits an immune response. Examples of an immunogen include, but are not limited to natural or synthetic (including modified) peptides, proteins, carbohydrates, lipids, oligonucleotides (RNA, DNA, etc.), chemicals, or other agents.

As used herein and in the claims, the term “polypeptide” encompasses amino acid chains of any length, including full-length proteins, wherein the amino acid residues are linked by covalent peptide bonds.

As used herein and in the claims, a “variant” is a polypeptide that differs from a native antigen only in conservative substitutions and/or modifications, such that antigenic properties of the native antigen are retained. Such variants may generally be identified by modifying a polypeptide sequence and evaluating the antigenic properties of the modified polypeptide. A “conservative substitution” is one in which an amino acid is substituted for another amino acid that has similar properties. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, be modified by, for example, the deletion or addition of amino acids that have minimal influence on the antigenic properties or structural characteristics of the polypeptide.

As used herein and in the claims, a “fusion protein” or “fusion polypeptide” comprises two or more protein/polypeptide sequences joined via a peptide linkage into a single amino acid chain. The sequences may be joined directly, without intervening amino acids, or by way of a linker amino acid sequence.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivations thereof, refers to an agent that modifies the effect of another agent while having few, if any, direct effects when given by itself. For example, an adjuvant may increase the potency or efficacy of a pharmaceutical, or an adjuvant may alter or affect an immune response.

As used herein and in the claims, the term “agonist” refers to a compound that can combine with a receptor (e.g., a Toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise resulting in the modification of a compound so that it directly binds to the receptor.

As used herein and in the claims, the term “antagonist” refers to a compound that can combine with a receptor (e.g., a Toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the terms “vehicle,” “carrier,” “pharmaceutical vehicle,” “pharmaceutical carrier,” “pharmaceutically acceptable vehicle,” “pharmaceutically acceptable carrier,” “diagnostic vehicle,” “diagnostic carrier,” “diagnostically acceptable vehicle,” or “diagnostically acceptable carrier” may be used interchangeably, depending on whether the use is pharmaceutical or diagnostic, and refer to pharmaceutically or diagnostically acceptable solid or liquid, diluting or encapsulating, filling or carrying agents, which are usually employed in pharmaceutical or diagnostic industry for making pharmaceutical or diagnostic compositions. Examples of vehicles include any liquid, gel, salve, cream, solvent, diluent, fluid ointment base, vesicle, liposomes, niosomes, ethasomes, transfersomes, virosomes, cyclic oligosaccharides, non ionic surfactant vesicles, phospholipid surfactant vesicles, micelle, and the like, that is suitable for use in contacting a subject.

In some embodiments, a pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacologically active agent in the formulation.

As used herein and in the claims, the term “cyclodextrin” refers to any of a family of cyclic oligosaccharides. Cyclodextrins, also sometimes called cycloamyloses, are composed of, but are not necessarily limited to, five or more D-glucopyranoside units, connected by α-(1,4) glycosidic linkages, as in amylase. Cyclodextrins having as many as 32 1,4-glucopyranoside units have been well characterized. Typically, cyclodextrins contain, but are not necessarily limited to, six to eight glucopyranoside units in a ring, commonly termed α-cyclodextrin (six units), β-cyclodextrin (seven units), and γ-cyclodextrin (eight units). These may be naturally occurring or produced synthetically.

As used herein and in the claims, “in conjunction with” and any derivations thereof refers to administration of an active agent, vehicle, carrier, and the like, simultaneously with, prior to, or subsequent to administration of a further active agent, vehicle, carrier, and the like.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

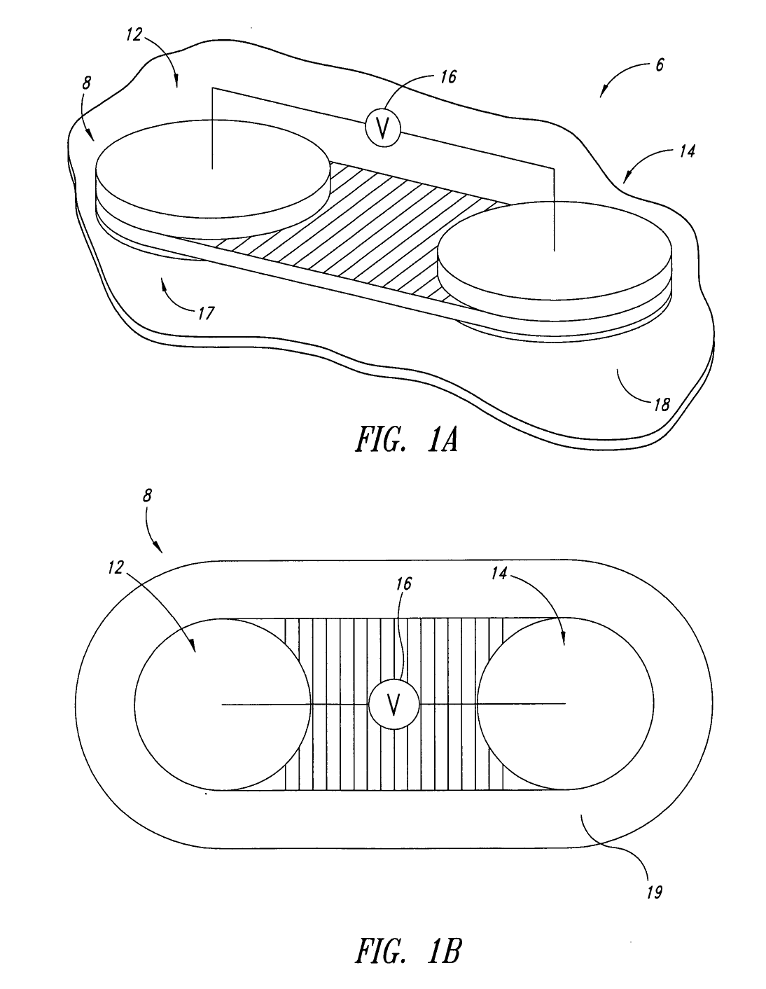

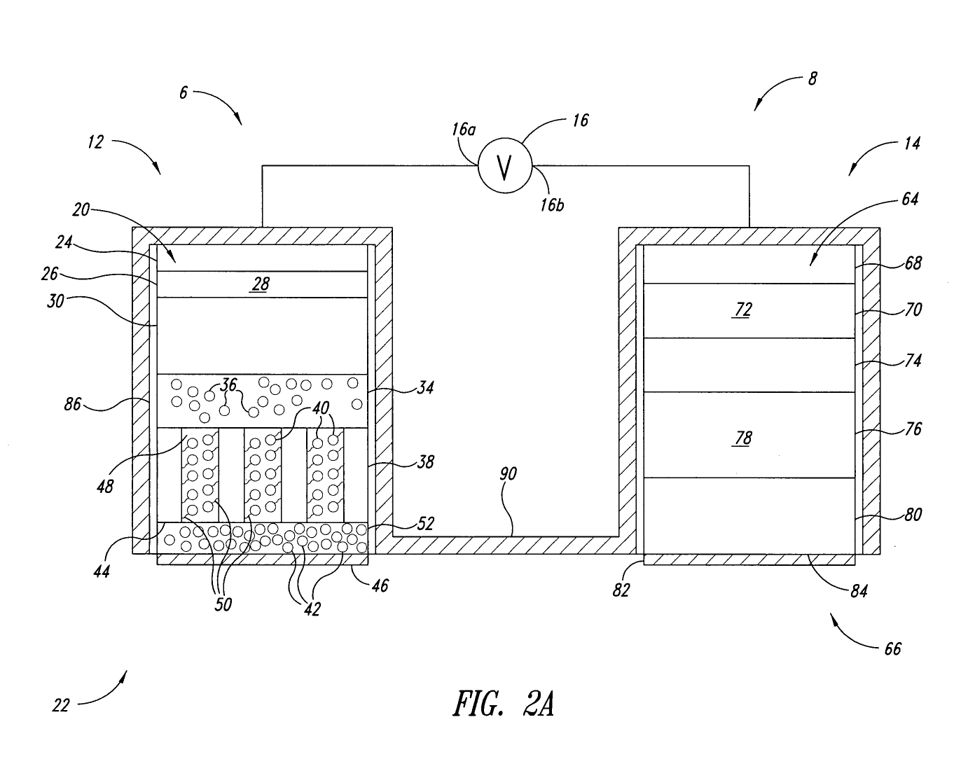

FIGS. 1A and 1B show an exemplary transdermal drug delivery system 6 for delivering of one or more active agents to a subject. The system 6 includes an iontophoresis device 8 including active and counter electrode assemblies 12, 14, respectively, and a power source 16. The active and counter electrode assemblies 12, 14, are electrically coupled to the power source 16 to supply an active agent contained in the active electrode assembly 12, via iontophoresis, to a biological interface 18 (e.g., a portion of skin or mucous membrane). In some embodiments, the iontophoresis device 8 may optionally include an outer adhesive surface 19 for physically coupling the iontophoresis device 8 to the biological interface 18 of the subject.

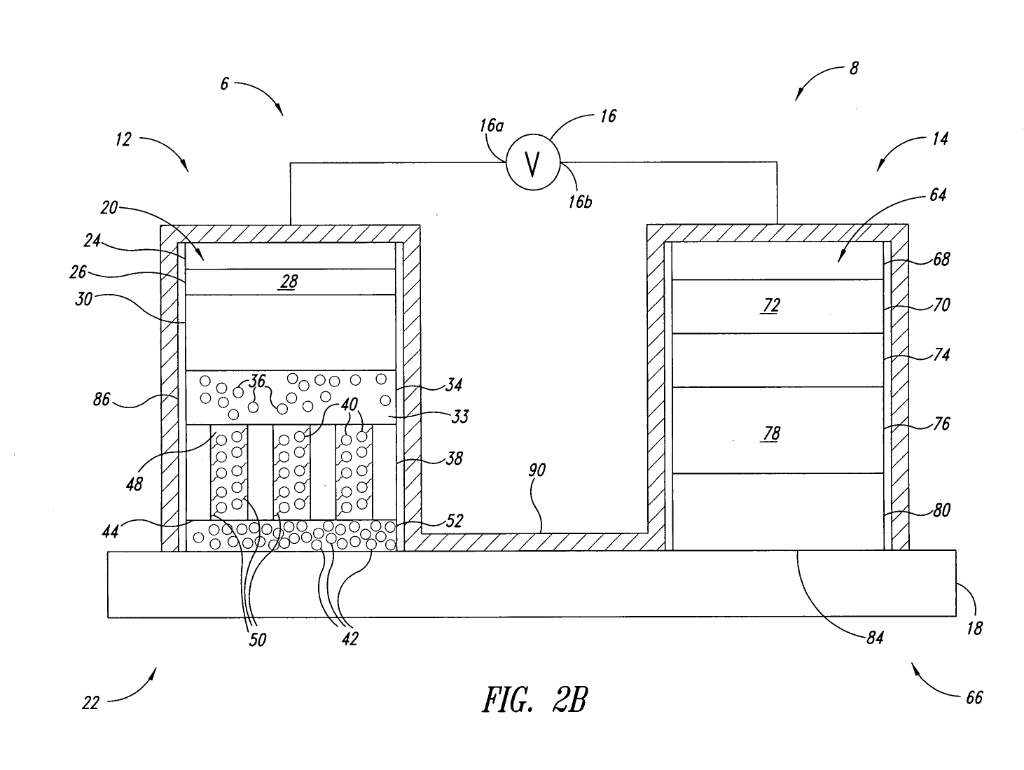

As shown in FIGS. 2A and 2B, the active electrode assembly 12 comprises, from an interior 20 to an exterior 22 of the active electrode assembly 12: an active electrode element 24, an electrolyte reservoir 26 storing an electrolyte 28, an inner ion selective membrane 30, an inner active agent reservoir 34 storing active agent 36, an optional outermost ion selective membrane 38 that optionally caches additional active agent 40, an optional further active agent 42 carried by an outer surface 44 of the outermost ion selective membrane 38, and an optional outer release liner 46.

The active electrode assembly 12 may further comprise an optional inner sealing liner (not shown) between two layers of the active electrode assembly 12, for example, between the inner ion selective membrane 30 and the inner active agent reservoir 34. The inner sealing liner, if present, would be removed prior to application of the iontophoretic device to the biological interface 18. Each of the above elements or structures will be discussed in detail below.

The active electrode element 24 is electrically coupled to a first pole 16a of the power source 16 and positioned in the active electrode assembly 12 to apply an electromotive force to transport the active agent 36, 40, 42 via various other components of the active electrode assembly 12. Under ordinary use conditions, the magnitude of the applied electromotive force is generally that required to deliver the one or more active agents according to a therapeutic or diagnostic effective dosage protocol. In some embodiments, the magnitude is selected such that it meets or may exceed the ordinary use operating electrochemical potential of the iontophoresis delivery device 8.

The active electrode element 24 may take a variety of forms. In one embodiment, the active electrode element 24 may advantageously take the form of a carbon-based active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such an anode includes an Ag/AgCl electrode. The reverse reaction takes place at the cathode in which the metal ion is reduced and the corresponding anion is released from the surface of the electrode.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities, or interstices, particularly where the electrolyte 28 is a liquid.

In one embodiment, the electrolyte 28 comprises ionic or ionizable components in an aqueous medium, which can act to conduct current towards or away from the active electrode element. Suitable electrolytes include, for example, aqueous solutions of salts. Preferably, the electrolyte 28 includes salts of physiological ions, such as sodium, potassium, chloride, and phosphate.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 28 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode, and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 28 may be in the form of an aqueous solution housed within a reservoir 26, or in the form of a dispersion in a hydrogel or hydrophilic polymer capable of retaining substantial amount of water. For instance, a suitable electrolyte may take the form of a solution of 0.5 M disodium fumarate: 0.5 M polyacrylic acid: 0.15 M anti-oxidant.

The inner ion selective membrane 30 is generally positioned to separate the electrolyte 28 and the inner active agent reservoir 34, if such a membrane is included within the device. The inner ion selective membrane 30 may take the form of a charge selective membrane. For example, when the active agent 36, 40, 42 comprises a cationic active agent, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na+) ions from the electrolyte 28, thereby increasing the transfer rate and/or biological compatibility of the iontophoresis device 8.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36. For example, the inner active agent reservoir 34 may take the form of a pouch or other receptacle, a membrane with pores, cavities, or interstices, particularly where the active agent 36 is a liquid. The inner active agent reservoir 34 further may comprise a gel matrix.

Optionally, an outermost ion selective membrane 38 is positioned generally opposed across the active electrode assembly 12 from the active electrode element 24. The outermost membrane 38 may, as in the embodiment illustrated in FIGS. 2A and 2B, take the form of an ion exchange membrane having pores 48 (only one called out in FIGS. 2A and 2B for sake of clarity of illustration) of the ion selective membrane 38 including ion exchange material or groups 50 (only three called out in FIGS. 2A and 2B for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane, thus allowing the passage of the cationic active agent while blocking the back flux of the anions present in the biological interface, such as skin. Alternatively, where the active agent 36, 40, 42 is an anion, the outermost ion selective membrane 38 may take the form of an anion exchange membrane, thus allowing the passage of anionic active agent.

The outermost ion selective membrane 38 may optionally cache active agent 40. Without being limited by theory, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of a semi-permeable or microporous membrane that is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be optionally preloaded with the additional active agent 40, such as ionized or ionizable drugs or therapeutic or diagnostic agents and/or polarized or polarizable drugs or therapeutic or diagnostic agents. Where the outermost ion selective membrane 38 is an ion exchange membrane, a substantial amount of active agent 40 may bond to ion exchange groups 50 in the pores, cavities, or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness, or coverage as to constitute a layer in a conventional sense of such term.

The active agent 42 may be deposited in a variety of highly concentrated forms such as, for example, solid form, nearly saturated solution form, or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiments, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives.

An interface-coupling medium (not shown) may be employed between the electrode assembly and the biological interface 18. The interface-coupling medium may, for example, take the form of an adhesive and/or gel. The gel may, for example, take the form of a hydrating gel. Selection of suitable bioadhesive gels is within the knowledge of one skilled in the relevant art.

In the embodiment illustrated in FIGS. 2A and 2B, the counter electrode assembly 14 comprises, from an interior 64 to an exterior 66 of the counter electrode assembly 14: a counter electrode element 68, an electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupled to a second pole 16b of the power source 16, the second pole 16b having an opposite polarity to the first pole 16a. In one embodiment, the counter electrode element 68 is an inert electrode. For example, the counter electrode element 68 may take the form of the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen or oxygen, depending on the polarity of the electrode) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxyl (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane. Typically, the outermost ion selective membrane 80 of the counter electrode assembly 14 is selective to ions with a charge or polarity opposite to that of the outermost ion selective membrane 38 of the active electrode assembly 12. The outermost ion selective membrane 80 is therefore an anion exchange membrane, which substantially passes anions and blocks cations, thereby prevents the back flux of the cations from the biological interface. Examples of suitable ion exchange membranes are discussed above.

Alternatively, the outermost ion selective membrane 80 may take the form of a semi-permeable membrane that substantially passes and/or blocks ions based on size or molecular weight of the ion.

The outer release liner 82 may generally be positioned overlying or covering an outer surface 84 of the outermost ion selective membrane 80. The outer release liner 82 is shown in place in FIG. 2A and removed in FIG. 2B. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The iontophoresis device 8 may further comprise an inert molding material 186 adjacent exposed sides of the various other structures forming the active and counter electrode assemblies 12, 14. The molding material 86 may advantageously provide environmental protection to the various structures of the active and counter electrode assemblies 12, 14. Enveloping the active and counter electrode assemblies 12, 14 is a housing material 90.

As best seen in FIG. 2B, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In use, the outermost active electrode ion selective membrane 38 may be placed directly in contact with the biological interface 18. Alternatively, an interface-coupling medium (not shown) may be employed between the outermost active electrode ion selective membrane 22 and the biological interface 18. The interface-coupling medium may, for example, take the form of an adhesive and/or gel. The gel may, for example, take the form of a hydrating gel or a hydrogel. If used, the interface-coupling medium should be permeable by the active agent 36, 40, 42.

In some embodiments, the power source 16 is selected to provide sufficient voltage, current, and/or duration to ensure delivery of the one or more active agents 36, 40, 42 from the reservoir 34 and across a biological interface (e.g., a membrane) to impart the desired physiological effect. The power source 16 may take the form of one or more chemical battery cells, super- or ultra-capacitors, fuel cells, secondary cells, thin film secondary cells, button cells, lithium ion cells, zinc air cells, nickel metal hydride cells, and the like. The power source 16 may, for example, provide a voltage of 12.8 V DC, with tolerance of 0.8 V DC, and a current of 0.3 mA. The power source 16 may be selectively electrically coupled to the active and counter electrode assemblies 12, 14 via a control circuit, for example, via carbon fiber ribbons. The iontophoresis device 8 may include discrete and/or integrated circuit elements to control the voltage, current and/or power delivered to the electrode assemblies 12, 14. For example, the iontophoresis device 8 may include a diode to provide a constant current to the electrode elements 24, 68.

As suggested above, the one or more active agents 36, 40, 42 may take the form of one or more cationic or an anionic drugs or other therapeutic or diagnostic agents. Consequently, the poles or terminals of the power source 16 and the selectivity of the outermost ion selective membranes 38, 80 and inner ion selective membranes 30, 74 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides or nucleic acids. In other embodiments, the active agent may be mixed with another agent to form a complex capable of being transported across the biological interface via one of the motive methods described above.

In some embodiments, the transdermal delivery system 6 includes an iontophoretic delivery device 8 for providing transdermal delivery of one or more therapeutic or diagnostic active agents 36, 40, 42 to a biological interface 18. The delivery device 8 includes active electrode assembly 12 including at least one active agent reservoir and at least one active electrode element operable to provide an electromotive force to drive an active agent from the at least one active agent reservoir. The delivery device 8 may include a counter electrode assembly 14 including at least one counter electrode element 68, and a power source 16 electrically coupled to the at least one active and the at least one counter electrode elements 24, 68. In some embodiments, the iontophoretic delivery device 8 may further include one or more active agents 36, 40, 42 loaded in the at least one active agent reservoir 34.

In certain embodiments, an iontophoretic device may be used in methods for the localized administration of one, or more than one, angiogenic growth factor, or a composition thereof, to, into or through a biological interface to promote and/or enhance healing or repair of injured or damaged tissue. Angiogenesis, the process of forming new blood vessels, plays a significant role in physiological events such as wound healing and tissue remodeling. After injury or insult to tissue, angiogenesis is instrumental in restoring blood flow to the site of injury or insult in order to supply a variety of nutrients and factors necessary for healing and rebuilding to occur. Angiogenesis is stimulated by any of a variety of growth factors. Therapeutic administration of one, or more than one, angiogenic growth factor, or a composition thereof, may assist in the healing and repair processes, particularly in patient populations in which healing and repair are impaired, for example, the elderly and/or those with diabetes.

Angiogenic growth factors, or compositions thereof, may be applied topically or systemically. Topically applied growth factors or compositions are primarily limited to treatment of small superficial areas at or closely proximate to a biological interface, such as a surface of an area of damage or injury. Systemic administration, wherein the growth factors circulate throughout the body, is a poor method for providing these factors to specific sites within the body and further may result in serious undesirable side effects. To overcome one or more of the shortcomings, and to provide further advantages, angiogenic growth factors, including, but not limited to, those identified herein, or compositions thereof, may be advantageously administered iontophoretically using the devices and methods disclosed and described herein.

Iontophoretic devices and materials and methods are provided for use in promoting and/or enhancing healing or repair of damaged or injured tissue in a subject. In certain embodiments, an iontophoresis device as disclosed herein may comprise one, or more than one, angiogenic growth factor, or a combination or composition thereof, useful for promoting and/or enhancing healing or repair of damaged or injured tissue, including for example healing of wounds, bedsores, or other ulcerative conditions. In some embodiments, the iontophoresis devices and methods are provided for localized administration of the one, or more than one, angiogenic growth factor, or combination or composition thereof, to, into or through a biological interface to promote and/or enhance healing or repair of injured or damaged tissue.

In certain embodiments, active agents 36 and/or 40 and/or 42 of the iontophoretic device, as in FIGS. 2A and 2B, may comprise one, or more than one, angiogenic growth factor, such as those identified herein, or a composition thereof, useful to promote or enhance repair of injured or damaged tissue. In certain embodiments, active agents 36, 40 and 42 may be identical to one another. In certain other embodiments, active agents 36, 40 and 42 may differ from one another. In certain aspects, for example, active agents 36, 40 and 42 may each comprise basic fibroblast growth factor (bFGF). In yet other aspects, for example, active agents 36, 40 and 42 may each comprise vascular endothelial growth factor (VEGF)/vascular permeability factor (VPF). In further aspects, for example, each of active agents 36, 40 and 42 may comprise either bFGF or VEGF/VPF. The iontophoretic devices and methods disclosed herein for the localized delivery or administration of one, or more than one, angiogenic growth factor, or a composition thereof, to, into or through a biological interface to promote and/or enhance healing or repair of injured or damaged tissue are in no manner limited by this exemplary disclosure of the use of bFGF and/or VEGF/VPF. As described elsewhere herein, a device according to the present disclosure may comprise only one, or two, or all three of active agents 36, 40, and 42.

In certain embodiments, an iontophoresis device comprising one, or more than one, angiogenic growth factor, or a composition thereof, may be applied to a biological interface. In some embodiments, the biological interface may be the surface of an injured or damaged tissue. In certain such embodiments, upon application of an electromotive force or current to the iontophoresis device, the one, or more than one, angiogenic growth factor, or composition thereof, may be administered to or into the injured or damaged tissue. In some other embodiments, the biological interface may be a skin, for example an area of skin overlying an injured or damaged tissue. In yet other aspects, the biological interface may be a surface of an organ, for example a heart.

In certain embodiments, a method for delivery of one, or more than one, angiogenic growth factor, or a composition thereof, to or into an injured or damaged tissue may comprise, for example, (1) positioning an iontophoretic delivery device comprising one, or more than one, angiogenic growth factor, or a composition thereof, on a surface of a biological interface, such as, for example, on a surface area of an injured or damaged tissue; (2) switching the device on to supply an electromotive force or current; and (3) allowing the one, or more than one, angiogenic growth factor, or a composition thereof, to be administered under the influence of the electromotive force or current to or into the injured or damaged tissue. In certain embodiments, the method may comprise electrically coupling an active electrode assembly and a counter electrode assembly to poles of a power source and activating the power source to apply an electromotive force or current to the active electrode assembly and the counter electrode assembly. In some embodiments, the iontophoretic delivery device may be applied to a site of injury or insult, for example to an ulcerated area that has breached the skin. In some other embodiments, the device may be applied, for example, to an area, or a portion of an area, surrounding a site of injured or damaged tissue. Under the influence of the electromotive force or current supplied by the power source, when the device is switched on, the one, or more than one, angiogenic growth factor, or a composition thereof, may be delivered directly to or into an injured or damaged tissue. Alternatively, or additionally, the one, or more than one, angiogenic growth factor, or a composition thereof, may be delivered through a surface of a biological interface surrounding and/or covering an injured or damaged area.

In certain aspects, the iontophoretic device comprising one, or more than one, angiogenic growth factor, or a composition thereof, may be set to establish a magnitude of electromotive force or current to be supplied by the power source 16 for a fixed time so as to deliver the one, or more than one, angiogenic growth factor to, proximate to, or into the damaged or injured tissue. In certain such aspects, the magnitude of the electromotive force or current and/or the duration of the delivery may be a pre-set characteristic of the device. In certain other aspects, the magnitude of the electromotive force or current and/or the duration of time may be set manually. In certain such aspects, upon activation of the device, the electromotive force or current is applied for the fixed time to transport the one, or more than one, angiogenic growth factor, or composition thereof, to or into the damaged or injured tissue. The duration of time during which the electromotive force or current is applied and/or the magnitude of electromotive force or current may be set to control the depth to which the one, or more than one, active agent, or composition thereof, is delivered, depending on the location, depth, and nature of the injured or damaged tissue.

In further embodiments, an electromotive force or current may be set to deliver the one, or more than one, active agent, or a composition thereof, continuously at low levels to provide a sustained angiogenic effect at a site of an injured or damaged tissue. In certain other embodiments, an electromotive force or current may be applied in a pulsed manner over a sustained period of time to further control the dose of active agent(s) administered. In yet other embodiments, an electromotive force or current may be applied in a pulsed manner to control the depth to which the one, or more than one, active agent, or composition thereof, is administered.

In certain embodiments, an iontophoretic device accordingly to the present disclosure may be used to administer a single dose of one, or more than one, angiogenic growth factor, or composition thereof. In other embodiments, the device may be use to administer multiple repeat doses at different times, such as daily or weekly, in order to optimize healing or repair of the injured or damaged tissue.

The contact area of the device, that is, the area that contacts the biological interface of the subject, may vary in size. Surface area of an injured or damaged tissue may vary greatly. Accordingly, a variety of devices of different sizes may be available to allow selection of a device with a size approximating that of the injured or damaged tissue. In certain aspects, the size of the device may be such that the surface of the device contacting the biological interface may contact the entire area of the injured or damaged tissue and portions of surrounding areas. In other aspects, the surface of the device contacting the biological interface may contact only the area of the injured or damaged tissue. In yet other aspects, the surface of the device may contact only portions of the area of the injured or damaged tissue.

In certain embodiments, the iontophoretic device according to the present disclosure may further comprise one, or more than one, further active agent in addition to the one, or more than one, angiogenic growth factor, or composition thereof. In certain such embodiments or in further embodiments, the device may comprise agents that serve to enhance the ability of the angiogenic growth factor(s) to promote healing and/or repair of injured or damaged tissue. In yet other embodiments, a device may comprise agents that may have advantageous therapeutic effects, different from but complementary to those of the angiogenic growth factor(s), on an area of injured or damaged tissue. In certain embodiments, such agents may include, for example, anti-infective or anti-bacterial agents.

In certain embodiments, delivery of the one, or more than one, angiogenic growth factor, or a composition thereof, may occur primarily by electroosmotic flow, as disclosed elsewhere herein.

The above description of illustrated embodiments, including what is described in the Abstract, is not intended to be exhaustive or to limit the claims to the precise forms disclosed. Although specific embodiments and examples are described herein for illustrative purposes, various equivalent modifications can be made without departing from the spirit and scope of the invention, as will be recognized by those skilled in the relevant art. The teachings provided herein can be applied to other agent delivery systems and devices, not necessarily the exemplary iontophoresis active agent system and devices generally described above. For instance, some embodiments may omit one or more reservoirs, membranes or other structure. In other instances, some embodiments may include additional structure. For example, some embodiments may include a control circuit or subsystem to control a voltage, current, or power applied to the active and counter electrode elements 24, 68. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd. In certain embodiments, preparations of these various hydrogels may be made to incorporate proteins or polypeptides, or fusion proteins or fusion polypeptides, for use with the devices and methods disclosed herein. In certain embodiments, such hydrogel preparations may serve as reservoirs for the various active agents. Such hydrogel preparations may constitute, for example, inner active agent reservoir 34 or layer 52 of the active electrode assembly in FIGS. 2A and 2B.

Various embodiments discussed herein may advantageously employ microstructures, for example, microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids. Microneedle devices may be used, for example, to deliver any of a variety of compounds and/or compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having a solution of an active agent, such as a drug or therapeutic or diagnostic agent, that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the voltage source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the voltage source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No. WO03037425; U.S. patent application Ser. No. 10/488970, filed Mar. 9, 2004; Japanese patent application 2004/317317, filed Oct. 29, 2004; U.S. provisional patent application Ser. No. 60/627,952, filed Nov. 16, 2004; Japanese patent application Ser. No. 2004-347814, filed Nov. 30, 2004; Japanese patent application Ser. No. 2004-357313, filed Dec. 9, 2004; Japanese patent application Serial No. 2005-027748, filed Feb. 3, 2005; and Japanese patent application Serial No. 2005-081220, filed Mar. 22, 2005.

As one skilled in the relevant art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An iontophoretic delivery device, comprising: one or more angiogenic growth factors, or a composition thereof, wherein the one or more angiogenic growth factors, or the composition thereof, is useful to promote or enhance repair or healing of an injured or damaged tissue, and an electrode selectively operable to drive the one or more angiogenic growth factors, or the composition thereof, from the iontophoretic delivery device.

2. The device of claim 1 wherein the device is useful for localized administration of the one or more angiogenic growth factors, or composition thereof, to or into an area of injured or damaged tissue.

3. The device of claim 2 wherein the injured or damaged tissue is a wound, an ulcerated tissue or a bedsore.

4. The device of claim 2 wherein the one or more angiogenic growth factors, or composition thereof, comprises one or more growth factors selected from platelet-derived growth factor-BB (PDGF-BB), platelet-derived endothelial growth factor (PD-EDGF), angiogenin, angiopoietin-1, angiotropin, acidic fibroblast growth factor (aFGF), basic fibroblast growth factor (bFGF), Del-1, follistatin, granulocyte colony-stimulating factor (G-CSF), hepatocyte growth factor, interleukin-8 (IL-8), leptin, midkine, placental growth factor, pleiotrophin, progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

5. The device of claim 4 wherein the one or more angiogenic growth factors, or composition thereof, comprises one or more growth factors selected from platelet-derived growth factor-BB (PDGF-BB), platelet-derived endothelial growth factor (PD-EDGF), angiotropin, basic fibroblast growth factor (bFGF), granulocyte colony-stimulating factor (G-CSF), interleukin-8 (IL-8), pleiotrophin, progranulin, proliferin, transforming growth factor-alpha (TGF-alpha), transforming growth factor-beta (TGF-beta), and vascular endothelial growth factor (VEGF)/vascular permeability factor (VPF).

6. The device of claim 5 wherein the one or more angiogenic growth factors, or composition thereof, comprises one or more growth factors selected from the group consisting of basic fibroblast growth factor (bFGF) and vascular endothelial growth factor (VEGF)/vascular permeability factor (VPF).

7. A method for delivering an active agent to or through a biological interface of a subject, using an iontophoretic device comprising an active electrode assembly and a counter electrode assembly, the method comprising:
physically coupling the active electrode assembly and the counter electrode assembly to the biological interface of the subject; and
electrically coupling the active electrode assembly and the counter electrode assembly to poles of a power source to apply a voltage or current to the active electrode assembly and the counter electrode assembly,
wherein the electromotive force or current is applied to transport the active agent to or through the biological interface, and
wherein the active agent comprises one or more angiogenic growth factors, or a composition thereof, useful for promoting or enhancing repair or healing of an injured or damaged tissue.

8. The method of claim 7 wherein the one or more angiogenic growth factors, or composition thereof, comprises one or more growth factors selected from platelet-derived growth factor-BB (PDGF-BB), platelet-derived endothelial growth factor (PD-EDGF), angiogenin, angiopoietin-1, angiotropin, acidic fibroblast growth factor (aFGF), basic fibroblast growth factor (bFGF), Del-1, follistatin, granulocyte colony-stimulating factor (G-CSF), hepatocyte growth factor, interleukin-8 (IL-8), leptin, midkine, placental growth factor, pleiotrophin, progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

9. The method of claim 8 wherein the one or more angiogenic growth factors, or composition thereof, comprises one or more growth factors selected from platelet-derived growth factor-BB (PDGF-BB), platelet-derived endothelial growth factor (PD-EDGF), angiotropin, basic fibroblast growth factor (bFGF), granulocyte colony-stimulating factor (G-CSF), interleukin-8 (IL-8), pleiotrophin, progranulin, proliferin, transforming growth factor-alpha (TGF-alpha), transforming growth factor-beta (TGF-beta), and vascular endothelial growth factor (VEGF)/vascular permeability factor (VPF).

10. The method of claim 9 wherein the one or more angiogenic growth factors, or composition thereof, comprises one or more growth factors selected from basic fibroblast growth factor (bFGF) and vascular endothelial growth factor (VEGF)/vascular permeability factor (VPF).

11. A method of promoting or enhancing repair or healing of injured or damaged tissue using the device of claim 2, the method comprising:
positioning the device on a surface of an area of injured or damaged tissue;
switching the device on to supply an electromotive force or current; and
delivering the one or more angiogenic growth factors, or a composition thereof, to or into the injured or damaged tissue under the influence of the electromotive force or current.

12. The method of claim 11 wherein the one or more angiogenic growth factors, or composition thereof, comprises one or more growth factors selected from platelet-derived growth factor-BB (PDGF-BB), platelet-derived endothelial growth factor (PD-EDGF), angiogenin, angiopoietin-1, angiotropin, acidic fibroblast growth factor (aFGF), basic fibroblast growth factor (bFGF), Del-1, follistatin, granulocyte colony-stimulating factor (G-CSF), hepatocyte growth factor, interleukin-8 (IL-8), leptin, midkine, placental growth factor, pleiotrophin, progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).
